(19) United States
(12) Patent Application Publication
(10) Pub. No.: US 2021/0146083 A1
(43) Pub. Date: May 20, 2021

(54) RESPIRATORY MASK

(71) Applicant: HSINER CO., LTD., Taichung City (TW)

(72) Inventor: Eric CHANG, Taichung City (TW)

(73) Assignee: HSINER CO., LTD., Taichung City (TW)

(21) Appl. No.: 16/807,699

(22) Filed: Mar. 3, 2020

(30) Foreign Application Priority Data
Nov. 18, 2019 (TW) 108141785

(51) Int. Cl. A61M 16/06; A61M 16/08

(52) U.S. Cl. CPC A61M 16/0605; A61M 2205/3331; A61M 16/0875

(57) ABSTRACT

A respiratory mask includes an air duct, a muting member and a mask body. The muting member includes an annular portion sleeved on the air duct and formed with multiple muting holes extending therethrough, and a securing portion. The mask body is made of a flexible material, and has a connecting portion secured to the securing portion, and a groove portion connected to the connecting portion and defining a deformation groove that surrounds a central axis and that has a groove opening which faces parallel to the central axis. A ratio of an inner radius of the mask body to a groove width of the deformation groove ranges from 4 to 8. A ratio of the groove width to a groove depth of the deformation groove ranges from 0.5 to 1.5.

### CROSS-REFERENCE TO RELATED APPLICATION

This application claims priority of Taiwanese Invention Patent Application No. 108141785, filed on Nov. 18, 2019.

### FIELD

The disclosure relates to a medical mask, more particularly to a continuous positive airway pressure (CPAP) mask.

### BACKGROUND

Sleep is an important part of life for humans. Sleep deprivation may have negative impacts on a person's concentration, immune system, energy and memory. Patients of a type of sleep apnea known as obstructive sleep apnea (OSA) experience complete or partial obstruction of the upper airway by the tongue or other muscles during sleep, which causes hypoxia. OSA has numerous serious impacts on daily life of a patient, and in serious cases may lead to death. A common treatment for OSA is continuous positive airway pressure (CPAP) therapy which involves applying an air pressure on a continuous basis to keep the airway open while the patient is sleeping.

In order to apply this air pressure, a flow generator delivers air into a mask covering the mouth and nose of the patient. A conventional respiratory mask includes a mask body and an air duct connected between the mask body and the flow generator. A drawback of the conventional respiratory mask lies in that when the patient moves relative to the flow generator, the air duct also moves relative to the patient and often causes the mask body to change position relative to the face of the patient, which may result in large gaps between the mask body and the face of the patient. These gaps may have adverse effects on the application of the air pressure to the airway of the patient. Another drawback of the conventional respiratory mask is that exhaled gas of the patient is discharged through a gap between the mask body and the face of the patient, which may result in noise and negatively impact the user experience.

### SUMMARY

Therefore, the object of the disclosure is to provide a respiratory mask that can alleviate at least one of the drawbacks of the prior art.

According to the disclosure, an embodiment of a respiratory mask includes an air duct, a muting member and a mask body.

The air duct includes a duct segment, and an installation segment connected to the duct segment and extending along a central axis.

The muting member includes an annular portion and a securing portion. The annular portion surrounds the central axis, is sleeved on the installation segment of the air duct, and is formed with a plurality of muting holes extending therethrough. The securing portion is disposed at a side of the annular portion which is opposite to the installation segment of the air duct and is connected to the annular portion.

The mask body is made of a flexible material and has a connecting portion secured to the securing portion of the muting member, a groove portion surrounding and connected to the connecting portion, and defining a deformation groove that surrounds the central axis and that has a groove opening which faces parallel to the central axis, and a supporting portion surrounding and connected to the groove portion.

The mask body has an inner radius between the central axis and a junction between the groove portion and the connecting portion. The deformation groove has a groove width in a direction which is transverse to the central axis, and a groove depth in a direction of the central axis. A ratio of the inner radius to the groove width ranges from 4 to 8, and a ratio of the groove width to the groove depth ranges from 0.5 to 1.5.

### BRIEF DESCRIPTION OF THE DRAWINGS

Other features and advantages of the disclosure will become apparent in the following detailed description of the embodiment with reference to the accompanying drawings, of which:

FIG. 1 is a perspective view of an embodiment of a respiratory mask according to the disclosure;

FIG. 2 is an exploded perspective view of the embodiment;

FIG. 3 is a sectional view of the embodiment; and

FIG. 4 is a fragmentary, enlarged view of FIG. 3.

### DETAILED DESCRIPTION

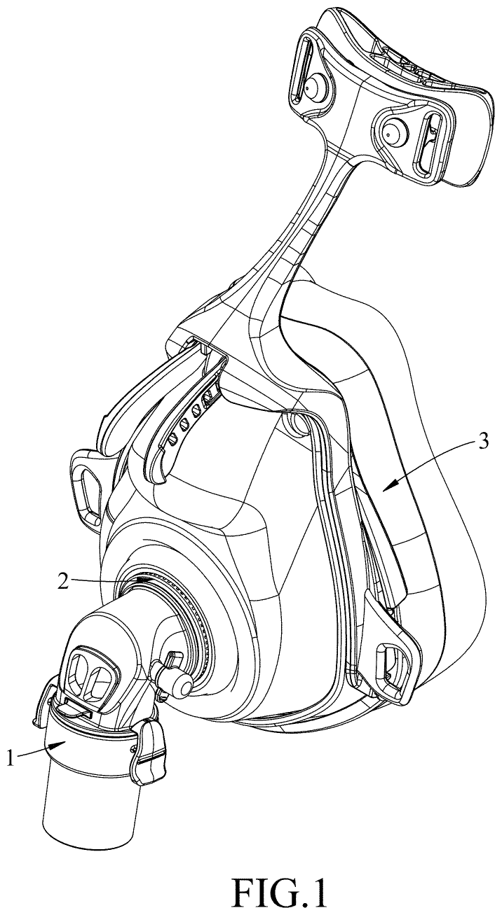

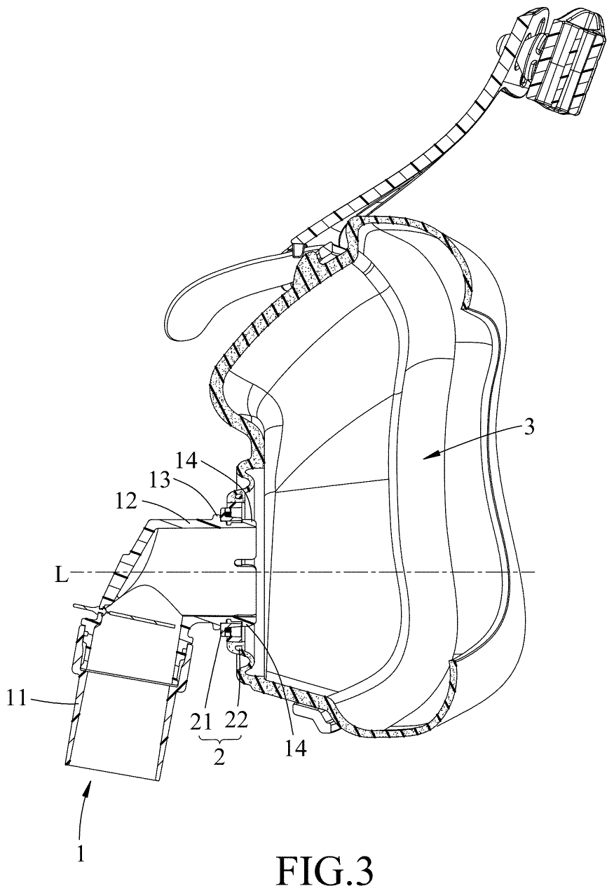

Referring to FIGS. 1 to 3, an embodiment of a respiratory mask according to the disclosure includes an air duct 1, a muting member 2, and a mask body 3.

The air duct 1 includes a duct segment 11, an installation segment 12 connected to the duct segment 11 and extending along a central axis (L), a mounting ring 13 surrounding and connected to the installation segment 12, and a plurality of mounting protrusions 14 angularly spaced apart from each other and protruding outwardly from the installation segment 12. The installation segment 12 is made of a flexible material. The mounting ring 13 is spaced apart from the mounting protrusions 14 along the central axis (L). The mounting ring 13 and the mounting protrusions 14 are respectively proximate to and distal from the duct segment 11.

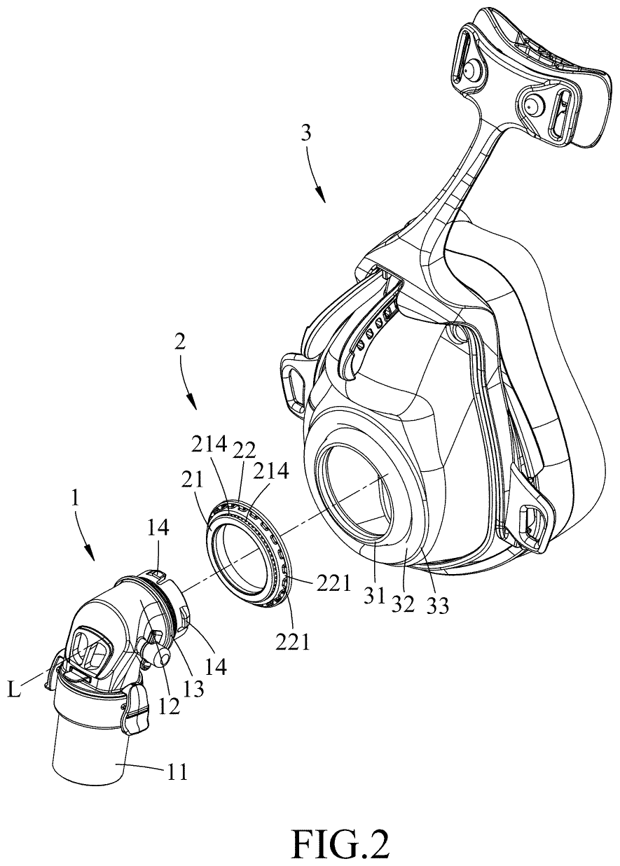

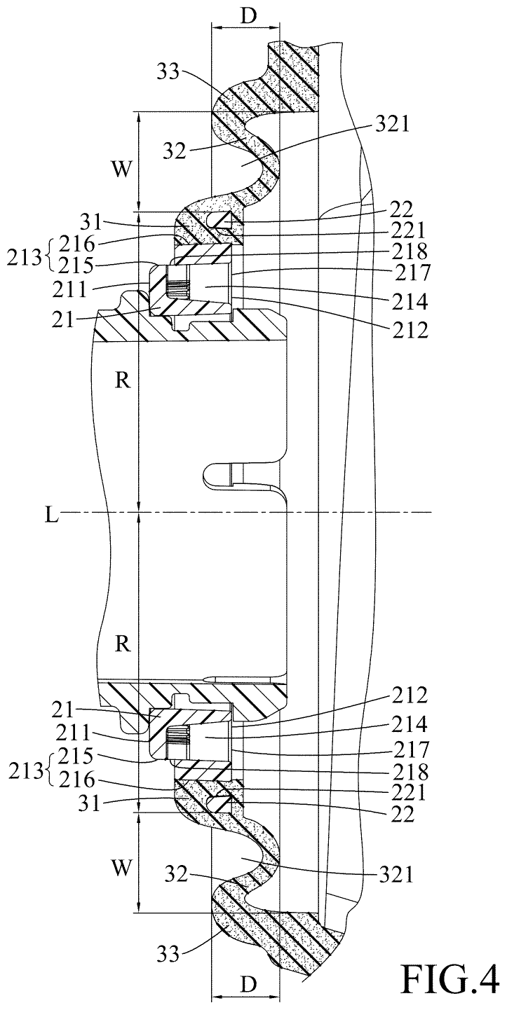

Referring to FIGS. 2 to 4, the muting member 2 includes an annular portion 21 and a securing portion 22.

The annular portion 21 surrounds the central axis (L), is sleeved on the installation segment 12 of the air duct 1, and is formed with a plurality of muting holes 214 extending therethrough and angularly spaced apart from each other. The annular portion 21 has outer and inner surfaces 211, 212 and an outer peripheral surface 213. The outer and inner surfaces 211, 212 are opposite to each other in the direction of the central axis (L) and are respectively proximate to and distal from the duct segment 11 of the air duct 1. The outer peripheral surface 213 surrounds the central axis (L), interconnects distal ends of the outer and inner surfaces 211, 212 which are opposite to the central axis (L), and is mounted with the securing portion 22. The outer peripheral surface 213 is stepped and has first and second sections 215, 216 respectively proximal to and distal from the duct segment 11 of the air duct 1. Each of the muting holes 214 has an inner opening 217 that is formed in the inner surface 212 of the annular portion 21 and an outer opening 218 that is formed in the first section 215 of the outer peripheral surface 213.

The securing portion 22 is disposed at a side of the annular portion 21 which is opposite to the installation segment 12 of the air duct 1, and is connected to the annular portion 21. Specifically, the securing portion 22 surrounds the annular portion 21 and is directly connected to the second section 216 of the outer peripheral surface 213. The securing portion 22 has a plurality of securing holes 221 extending therethrough and being parallel to the central axis (L).

The mask body 3 is made of a flexible material and has a connecting portion 31, a groove portion 32 and a supporting portion 33. The connecting portion 31 is secured to the securing portion 22 of the muting member 2. Specifically, the securing holes 221 of the securing portion 22 are engaged with the connecting portion 31. The groove portion 32 surrounds and is connected to the connecting portion 31, and defines a deformation groove 321 surrounding the central axis (L) and having a groove opening that faces parallel to the central axis (L). The supporting portion 33 surrounds and is connected to the groove portion 32.

In this embodiment, the mask body 3 is made of silicone, and a portion of the connecting portion 31 of the mask body 3 fills the securing holes 221 of the securing portion 22 during production and solidifies so that the connecting portion 31 of the mask body 3 and the securing portion 22 of the muting member 2 are secured to each other, but the method for connecting the mask body 3 to the muting member 2 is not limited in this respect.

The mask body 3 has an inner radius (R) between the central axis (L) and a junction between the groove portion 32 and the connecting portion 31. The deformation groove 321 has a groove width (W) in a direction which is transverse to the central axis (L), and a groove depth (D) in a direction of the central axis (L). A ratio of the inner radius (R) to the groove width (W) ranges from 4 to 8, and a ratio of the groove width (W) to the groove depth (D) ranges from 0.5 to 1.5. In this embodiment, a ratio of the inner radius (R) to the groove width (W) is substantially equal to 6 and a ratio of the groove width (W) to the groove depth (D) is substantially equal to 1.

In this embodiment, the deformation groove 321 has a curved cross-sectional outline. The groove depth (D) of the deformation groove 321 is the distance measured in the direction of the central axis (L) from an end of the cross-sectional outline which is proximal to the supporting portion 33 to the groove bottom of the cross-sectional outline. The groove width (W) is the distance measured in the direction perpendicular to the central axis (L) from the end of the cross-sectional outline which is proximal to the supporting portion 33 to an end of the cross-sectional outline which is proximal to the connecting portion 31. In other embodiments, the shape of the cross-sectional outline of the deformation groove 321 is not limited thus and may be, for example, a V-shape.

To assemble the embodiment, the installation segment 12 is squeezed to be deformed and extended along the central axis (L) through the annular portion 21 of the muting member 2. Deformation of the installation segment 12 is possible because it is made of a flexible material. Once the installation segment 12 is extended through the annular portion 21 of the muting member 2, it may be released so as to allow the elastic resilience thereof to restore the installation segment 12 back to the original shape, so that the annular portion 21 is secured between the mounting ring 13 and the mounting protrusions 14 of the air duct 1. In this configuration, the air duct 1 is connected to the muting member 2 but remains free to rotate about the central axis (L).

Also in this configuration, when the air duct 1 is moved relative to the mask body 3, as the mask body 3 is made of a flexible material, the groove portion 32 can deform to allow the connecting portion 31 to co-move with the air duct 1 to a certain degree, with only the connecting portion 31 and the groove portion 32 being affected by certain range of movement of the air duct 1 relative to the mask body 3. In other words, when the connecting portion 31 moves with the air duct 1, the deformation groove 321 changes shape due to deformation of the groove portion 32, but the supporting portion 33 surrounding the groove portion 32 is less likely to deform or move, and a portion of the mask body 3 may remain tightly abutted against the face of the patient. Hence, the air duct 1 of this embodiment may have a larger range of motion without moving the portion of the mask body 3 abutting against the face of the patient.

Moreover, by provision of the muting holes 214, the exhaled gas of the patient may be exhausted out of the mask body 3 from the inner openings 217 through to the outer openings 218 of the muting holes 214 which reduces noise compared with the conventional respiratory mask.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A respiratory mask comprising:
an air duct including a duct segment, and an installation segment connected to said duct segment and extending along a central axis;
a muting member including
an annular portion that surrounds the central axis, that is sleeved on said installation segment of said air duct, and that is formed with a plurality of muting holes extending therethrough, and
a securing portion that is disposed at a side of said annular portion which is opposite to said installation segment of said air duct and that is connected to said annular portion; and
a mask body made of a flexible material and having
a connecting portion that is secured to said securing portion of said muting member,
a groove portion that surrounds and is connected to said connecting portion, and that defines a deformation groove surrounding the central axis and having a groove opening that faces parallel to the central axis, and
a supporting portion that surrounds and is connected to said groove portion;
wherein said mask body has an inner radius between the central axis and a junction between said groove portion and said connecting portion, said deformation groove having a groove width in a direction which is transverse to the central axis, and a groove depth in a direction of the central axis, a ratio of said inner radius to said groove width ranging from 4 to 8, a ratio of said groove width to said groove depth ranging from 0.5 to 1.5.

2. The respiratory mask as claimed in claim 1, wherein:
said installation segment of said air duct is made of a flexible material; and
said air duct further includes a mounting ring surrounding and connected to said installation segment, and a plurality of mounting protrusions being angularly spaced apart from each other and protruding outwardly from said installation segment, said mounting ring being spaced apart from said mounting protrusions along the central axis, said mounting ring and said mounting protrusions being respectively proximate to and distal from said duct segment.

3. The respiratory mask as claimed in claim 1, wherein:
said annular portion of said muting member has
outer and inner surfaces being opposite to each other in the direction of the central axis and being respectively proximate to and distal from said duct segment of said air duct, and
an outer peripheral surface surrounding the central axis, interconnecting distal ends of said outer and inner surfaces which are opposite to the central axis, and mounted with said securing portion; and
said muting holes are angularly spaced apart from each other, each of said muting holes having an inner opening that is formed in said inner surface of said annular portion and an outer opening that is formed in said outer peripheral surface.

4. The respiratory mask as claimed in claim 3, wherein said outer peripheral surface is stepped and has first and second sections respectively proximal to and distal from said duct segment of said air duct, said outer openings of said muting holes being formed in said first section of said outer peripheral surface, said securing portion of said muting member being directly connected to said second section of said outer peripheral surface.

5. The respiratory mask as claimed in claim 1, wherein said securing portion of said muting member surrounds said annular portion, and has a plurality of securing holes extending therethrough, being parallel to the central axis, and being engaged with said connecting portion of said mask body.

6. The respiratory mask as claimed in claim 1, wherein said ratio of said inner radius to said groove width is substantially equal to 6.

7. The respiratory mask as claimed in claim 1, wherein said ratio of said groove width to said groove depth is substantially equal to 1.
